(19) United States
(12) Patent Application Publication
(10) Pub. No.: US 2008/0097450 A1
(43) Pub. Date: Apr. 24, 2008

(54) Patella clamp

(75) Inventors: Jeffrey D. Brown, Warsaw, IN (US); Charles A. Baldridge, Warsaw, IN (US); Kim C. Bertin, Salt Lake City, UT (US); Toby N. Farling, Warsaw, IN (US); Adam M. Griner, Columbia City, IN (US); Robert A. Hodorek, Warsaw, IN (US)

Correspondence Address: John F. Hoffman, Esq.;BAKER & DANIELS LLP, 111 East Wayne Street, Suite 800, Fort Wayne, IN, 46802, US

(73) Assignee: Zimmer Technology, Inc.

(21) Appl. No.: 11/521,852

(22) Filed: Sep. 14, 2006

(51) Int. Cl. A61B 17/58; A61B 17/08

(52) U.S. Cl. 606/88; 606/151

(57) ABSTRACT

A patella clamp is provided for clamping a patella. The patella clamp includes a first clamp member engageable with the patella and a second clamp member mountable in opposing relative moving relationship to the first clamp member. The patella clamp may be used to perform a variety of procedures on a patella. In one embodiment, a third clamp member is alternatively mountable in place of the second clamp member to change the function of the patella clamp. The patella clamp optionally further includes a cut guide for guiding a cutter in a cut plane to resect a portion of the patella.

### FIELD OF THE INVENTION

The invention relates to a patella clamp for use in procedures performed on the patella during a knee replacement procedure.

### BACKGROUND

Degenerative and traumatic damage to the articular cartilage of the knee joint can result in pain and restricted motion. Prosthetic joint replacement is frequently utilized to alleviate pain and restore joint function. In this surgical procedure, any one or all of the articulating compartments of the joint may be repaired including: the medial femoral/tibial joint compartment, the lateral femoral/tibial joint compartment, and the patella/femoral joint compartment. During such a procedure, the damaged compartment or compartments of the joint are cut away and replaced with prosthetic components.

For example, the posterior, articulating, side of the patella may be cut away and replaced with a prosthetic patellar component that will articulate against the natural femur or a prosthetic femoral component. The posterior side of the patella has typically been removed utilizing saws or reamers. The saws or reamers may be applied to the posterior patella freehand while the surgeon estimates the appropriate resection level. Often a retractor is used to manipulate the patella. Some kind of resection guide is typically provided to steady the saw or reamer and allow control of the resection level.

For example, in a typical procedure, the patella is flipped, or everted, so that the posterior surface faces anteriorly and a “C”-shaped clamp is applied to the sides of the patella to hold it securely. A flat surface or slot then guides a saw blade to resect the posterior surface of the patella. Examples of this type of side-clamping resection guide may be found in U.S. Pat. No. 4,633,862 issued Jan. 6, 1987, to Petersen and similar devices in U.S. Pat. No. 5,108,401; U.S. Pat. No. 5,542,947; and U.S. Pat. No. 6,010,509.

In another common procedure, the patella is everted and a clamp is applied to the anterior and posterior sides of the patella. The clamp face adjacent the posterior side of the patella includes a central opening through which a reamer is guided to ream away the posterior surface of the patella. Examples of this type of face-clamping resection guide may be found in U.S. Pat. No. 5,129,907 issued Jul. 14, 1992, to Heldreth et al. and similar devices in U.S. Pat. No. 5,129,908; U.S. Pat. No. 5,380,332; U.S. Pat. No. 5,486,177; U.S. Pat. No. 5,520,692; U.S. Pat. No. 5,536,271; U.S. Pat. No. 5,575,793; U.S. Pat. No. 5,658,291; U.S. Pat. No. 5,716,360; U.S. Pat. No. 5,885,298; U.S. Pat. No. 5,941,884; U.S. Pat. No. 6,159,246; and U.S. Pat. No. 6,602,258.

Existing patellar instruments tend to be bulky and require extensive exposure of the patella such that they are unsuitable for a minimally invasive surgical approach. They have fixed clamp-jaw geometries designed for a specific approach to the patella. They require eversion of the patella and where used to guide cutting of the patella they typically either gauge the cut based on a “bone removed” or a “bone remaining” methodology.

### SUMMARY

The present invention provides a patella clamp for clamping a patella. The patella clamp includes a first clamp member engageable with the patella and a second clamp member mounted in opposing relative moving relationship to the first clamp member. The patella clamp may be used to perform a variety of procedures on a patella.

In one aspect of the invention, the patella clamp includes a first clamp member engageable with the patella and a second clamp member removably mountable in opposing relative moving relationship to the first clamp member. A third clamp member is alternatively mountable in place of the second clamp member to change the function of the patella clamp.

In another aspect of the invention, the patella clamp optionally further includes a cut guide for guiding a cutter in a cut plane to resect a portion of the patella.

In another aspect of the invention, the patella clamp includes a handpiece having first and second handles pivotably mounted together. The handles each include a grip end and a working end. An anterior clamp member including a base defining an anterior clamp face is mounted to the working end of the first handle in translating relationship and is pivotably connected to the working end of the second handle. A first posterior clamp member, including a base defining a posterior clamp face, is removably mounted to the working end of the first handle in opposing relationship to the anterior clamp member such that moving the grip ends of the first and second handles toward and away from one another causes the anterior clamp member to translate toward and away from the first posterior clamp member. A second posterior clamp member is intraoperatively interchangeable with the first posterior clamp member to modify the function of the patella clamp.

In another aspect of the invention, the patella clamp includes a handpiece having first and second handles pivotably mounted together. The handles each include a grip end and a working end. An anterior clamp member including a base defining an anterior clamp face is mounted to the working end of the first handle in translating relationship and is pivotably connected to the working end of the second handle. A posterior clamp member, including a base defining a posterior clamp face, is mounted to the working end of the first handle in opposing relationship to the anterior clamp member such that moving the grip ends of the first and second handles toward and away from one another causes the anterior clamp member to translate toward and away from the first posterior clamp member. A cut guide is mounted to the patella clamp and is able to guide a cutter transversely between the anterior and posterior clamp members to cut the patella. The cut guide and at least one of the anterior and posterior clamp members defines a scale between them indicating the position of the cut plane relative to at least one of the anterior and posterior clamp faces.

### BRIEF DESCRIPTION OF THE DRAWINGS

Various examples of the present invention will be discussed with reference to the appended drawings. These drawings depict only illustrative examples of the invention and are not to be considered limiting of its scope.

FIG. 1 is an exploded perspective view of a patella clamp according to the present invention;

FIG. 2 is a side sectional view of the patella clamp of FIG. 1;

FIG. 3 is a perspective view of a posterior clamp member of the patella clamp of FIG. 1;

FIG. 4 is a perspective view of a posterior clamp member of the patella clamp of FIG. 1;

FIG. 5 is a perspective view of a posterior clamp member of the patella clamp of FIG. 1;

FIG. 6 is a perspective view of a posterior clamp member of the patella clamp of FIG. 1;

FIG. 7 is a perspective view of a posterior clamp member of the patella clamp of FIG. 1;

FIG. 8 is a perspective view of a cutter guide of the patella clamp of FIG. 1;

FIG. 9 is a front elevation view of a cutter guide of the patella clamp of FIG. 1; and

FIG. 10 is a front elevation view of a cutter guide of the patella clamp of FIG. 1.

### DESCRIPTION OF THE ILLUSTRATIVE EXAMPLES

Embodiments of a patella clamp include a pair of relatively movable clamp members. At least one of the clamp members may be removably mounted to the resection guide to permit substitution of alternative clamp members to adapt the patella clamp to differing uses. For example, alternative clamp members may be provided to facilitate orienting the patella in the patella clamp relative to its posterior surface, to facilitate orienting the patella in the resection guide relative to its anterior surface, to protect a resected posterior patella surface during subsequent surgical steps, to protect a natural posterior patella surface during subsequent surgical steps, and/or to provide other functions. The removable clamp member may be mounted by way of a snap-fit attachment, threaded attachment, pinned attachment, bayonet attachment, and/or other suitable attachment mechanism.

The patella clamp may support the patella relative to a cut guide in a predetermined position such that the cut guide may guide a cutter in a cut plane to resect the posterior patella at a predetermined depth and in a predetermined orientation. The position of the cut guide relative to the patella may be adjustable. For example, the cut guide may be adjustably mounted to the patella clamp to permit adjustment of depth of cut and/or cut plane orientation. The cut guide may include a threaded adjustment, a ratchet adjustment, a sliding frictional adjustment, a hinged adjustment, and/or any suitable adjustment mechanism.

The patella clamp is useable in any surgical procedure on the patella utilizing any approach for exposure of the patella. However, the patella clamp is particularly suitable for use in a minimally invasive approach from the side of the knee in which the patella is not everted. The patella clamp may clamp onto the anterior and posterior surfaces of the patella with the handles pointing, for example, medially or laterally. It may be placed without dissecting the tissues on the anterior side of the patella. The patella clamp may be used to stabilize the patella and/or guide a cutter to resect the patella while the patella remains in its normal anatomic position. The patella clamp may be used during any step in the surgical procedure. For example, the patella may be resected prior to other bone cuts, after all other bone cuts, or at some point in between.

The patella clamp may include one or more scales for indicating linear measurement relative to a patella resection. For example, a scale may be provided that indicates the total patella thickness prior to resecting the patella. A scale may be provided that indicates the patella thickness remaining after the resection with a particular patella clamp and cut guide configuration. A scale may be provided that indicates the amount of patellar bone that will be removed with a particular patella clamp and cut guide configuration.

The cut guide may be configured to guide any suitable cutter such as a saw blade, side cutting rotary cutter, end cutting rotary cutter, and/or other cutters. For example, the cutter guide may define a surface, slot, notches, and/or other features for guiding a saw blade in a cutting plane.

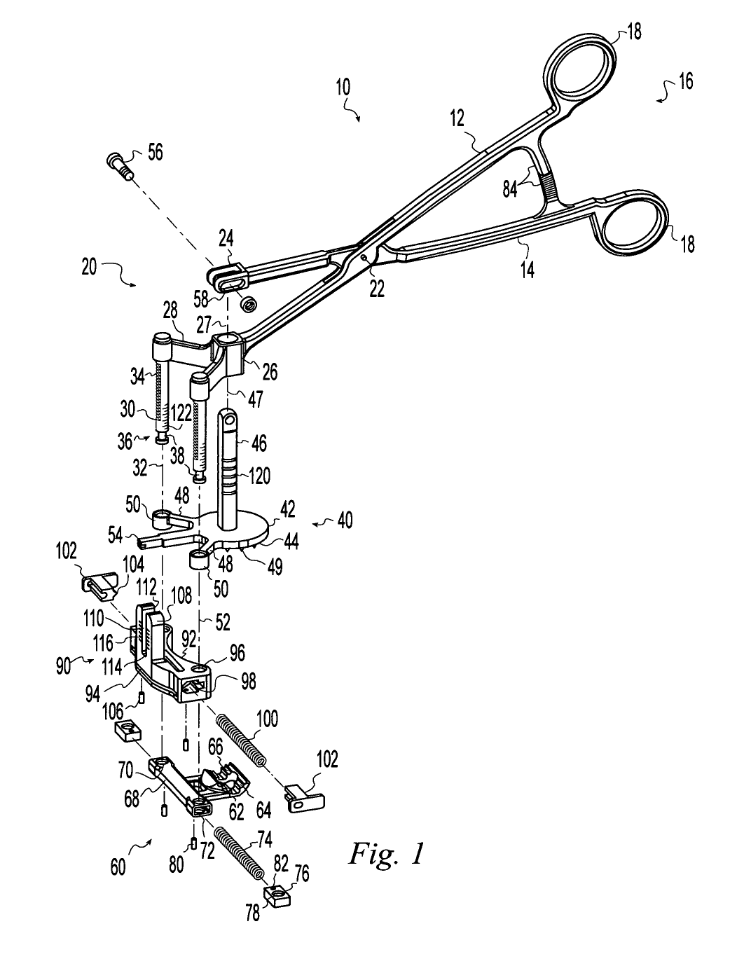

FIGS. 1-8 depict an illustrative patella clamp 10 including a handpiece having first 12 and second 14 handles pivotably mounted together. The handles 12, 14 include a grip end 16 defined by finger loops 18 and a working end 20. The handles 12, 14 are pinned together by a pivot pin 22 intermediate the grip end 16 and the working end 20. The working end 20 includes a clevis 24 defined by the second handle 14 and a sleeve 26, having a sleeve axis 27, defined by the first handle 12. An outrigger 28 extends from each of two sides of the sleeve 26 and each outrigger supports a guide rod 30 having a guide rod axis 32 parallel to the sleeve axis 27. Each guide rod 30 defines quick adjustment notches 34 and a quick attachment foot 36. The quick attachment foot 36 includes an annular groove 38.

An anterior clamp member 40 includes a base 42 defining a clamp face 44. A support rod 46 having a support rod axis 47 extends from the base 42 opposite the clamp face 44 generally perpendicular to the clamp face 44. In the illustrative embodiment, the anterior clamp face 44 is generally planar and includes a plurality of sharpened projections 49 for gripping the anterior side of the patella. An outrigger 48 extends from each of two sides of the anterior clamp member base 42 and each outrigger defines at its free end a sleeve 50 having a sleeve axis 52 parallel to the support rod axis 47. A pointer 54 extends from the base 42 intermediate the anterior clamp base sleeves 50. The anterior clamp 40 is assembled to the handles 12, 14 by inserting the support rod 46 into the sleeve 26 defined by the first handle 12 and inserting the guide rods 30 into the sleeves 50 defined by the anterior clamp member base 42. The anterior clamp member 40 is translatable up and down parallel to the sleeve axes 27, 52. The upper end of the support rod 46 inserts into the clevis 24 of the second handle 14 and is pinned in place by a pivot screw 56 through the clevis 24 and the support rod 46. The clevis 24 includes a transverse elongated through slot 58 to permit the pivot screw 56 to translate from side to side within the clevis 24 as the handles 12, 14 pivot relative to one another.

A modular, removable posterior clamp member 60 includes a base 62 defining a posterior clamp face 64. The posterior clamp face 64 includes a generally cylindrical concavity with elongated teeth 66 for gripping the posterior side of the patella. A support arm 68 is mounted to the base 62 and defines a quick attachment mechanism for permitting the posterior clamp member 60 to be quickly attached and removed from the guide rods 30. The quick attachment mechanism includes a hole 70 in the support arm 68 for receiving the quick attachment foot 36. A transverse opening 72 communicates with the hole 70 and receives a spring 74 and a button 76. The button 76 includes a first hole 78 for receiving the quick attachment foot 36. A retention pin 80 inserts through the bottom of the support arm 68 and presses into a second hole 82 in the button 76. The retention pin 80 slides in a slot (not shown) in the bottom of the support arm 68 to allow the button 76 to translate in and out of the opening 72 in a defined range of travel while preventing the button 76 from coming all the way out of the opening 72. The spring 74 biases the button outwardly. In the illustrative embodiment, a pair of opposing buttons 76 is provided. By pressing the buttons 76 inwardly against spring pressure, the holes 78 in the buttons may be aligned with the holes in the support arm 68 to receive the quick attachment foot 36. When the buttons are released, the buttons are biased outwardly such that a portion of each button 76 is pressed into the annular groove 38 of the quick attachment foot 36 to secure the posterior clamp member 60 to the guide rods 30. Similarly, by pressing the buttons 76 the posterior clamp member 60 may be quickly removed.

The patella clamp 10, with the handles 12, 14 and anterior and posterior clamp members 40, 60, may be used to grip and manipulate a patella during surgery. The posterior clamp face 64 may be positioned adjacent to the posterior side of the patella and the anterior clamp face 44 may be positioned adjacent to the anterior side of the patella. As the finger loops 18 of the handles 12, 14 are brought closer together, the anterior clamp member 40 is translated down relative to the first handle 12 to grip the patella between the anterior and posterior clamp faces 44, 64. A ratchet locking mechanism includes opposing ribbed tabs 84 that ratchet together to hold the handles 12, 14 in a clamped position. To release the handles 12, 14, the handles are flexed apart transverse to the tabs 84 to disengage the ribs.

A removable cut guide 90 may also be attached to the patella clamp 10 to guide a cutter to resect a portion of the patella. In the illustrative embodiment, the cut guide 90 includes a body 92 having a slot 94 defining a cut plane. The slot 94 is sized to receive a saw blade to guide the saw blade in the cut plane to resect the patella at a desired location. The cut guide 90 includes a quick adjustment mechanism including a pair of through holes 96 in the body 92 for receiving the guide rods 30. Transverse openings 98 communicate with the holes 96 and receive a spring 100 and buttons 102. Each button 102 includes a rearwardly facing tab 104 engageable with the quick adjustment notches 34 in the guide rods 30. Retention pins 106 insert through the bottom of the body 92 and press into the buttons 102. The retention pins 106 slide in slots (not shown) in the bottom of the body 92 to allow the buttons 102 to translate in and out of the openings 98 in a defined range of travel while preventing the buttons 102 from coming all the way out of the openings 98. The spring 100 biases the buttons outwardly. By pressing the buttons 102 inwardly against spring pressure, the tabs 104 are disengaged from the notches 34 and the cut guide 90 may be adjusted up and down on the guide rods 30 to position the slot 94 and consequently the cut plane to a desired position on the patella. When the buttons are released, the buttons are biased outwardly and the tabs 104 engaged the notches 34 thereby locking the vertical position of the cut guide 90 relative to the guide rods 30. The cut guide 90 may be removed from the patella clamp 10, by first removing the posterior clamp member 60 and then pressing the buttons 102 to disengage the notches 34 and sliding the cut guide 90 off of the guide rods 30.

Alternatively, at least one of the guide rods 30 may be threaded and at least one of the through holes 96 may be threaded such that by rotating the threaded guide rod 30, the cut guide 90 will translate up and down to adjust the cut guide position relative to the patella clamp.

The illustrative cut guide 90 includes a vertically extending wall 108 defining a vertical slot 110 having an open top end 112 and a closed bottom end 114. The slot 110 receives the pointer 54 in vertical translating relationship. The wall includes a scale 116 marked on its front face. With the patella clamped between the anterior and posterior clamp members 40, 60 and the cut guide 90 positioned on the guide rods 30, the pointer 54 is positioned adjacent to the scale 116. The pointer 54 and scale 116 indicate the distance from the anterior side of the patella to the cut plane and thus the amount of bone remaining after the patella is cut. In the illustrative cut guide 90, the optional closed bottom end 114 of the slot 110 abuts the pointer 54 to prevent the cut guide 90 from being adjusted closer than an established minimum distance from the anterior clamp face 44 and thus to establish a minimum remaining patella thickness. Alternatively, the sleeves 50 may be arranged to abut the cut guide body 92 to establish the minimum distance. A stop arrangement prevents a user from inadvertently removing too much of the patella. For example, there may be a minimum patellar thickness required to properly receive a patellar implant.

Other scales may also be provided. For example, in the illustrative embodiment, the support rod 46 includes a vertical scale 120 marked along its side. The posterior clamp member 60 attaches to the guide rods 30 at a fixed, predetermined distance from the sleeve 26 and the anterior clamp member 40 translates vertically relative to the sleeve 26. The scale 120 is marked to indicate the distance between the clamp faces 44, 64. Therefore, the scale 120 may be read to indicate the total thickness of the patella when it is clamped between the anterior and posterior clamp faces 44, 64. In the illustrated embodiment, the scale 120 is read relative to the top of the sleeve 26 in which the support rod 46 translates.

Another scale may be provided. For example, in the illustrative embodiment, the guide rods 30 include a vertical scale 122 marked along their sides. The scale 122 is marked to indicate the distance from the posterior clamp face 64 to the cut plane. Therefore, the scale may be read to indicate the distance from the posterior side of the patella to the cut plane and thus the amount of bone removed when the patella is cut. In the illustrative embodiment, the scale 122 is read relative to the bottom of the cut guide 90.

Any one of these scales, any combination of them, or all of them may be provided on the patella clamp 10 of the present invention. Preferably, all of the scales are provided to maximize the ability of the patella clamp 10 to be used in a variety of surgical techniques.

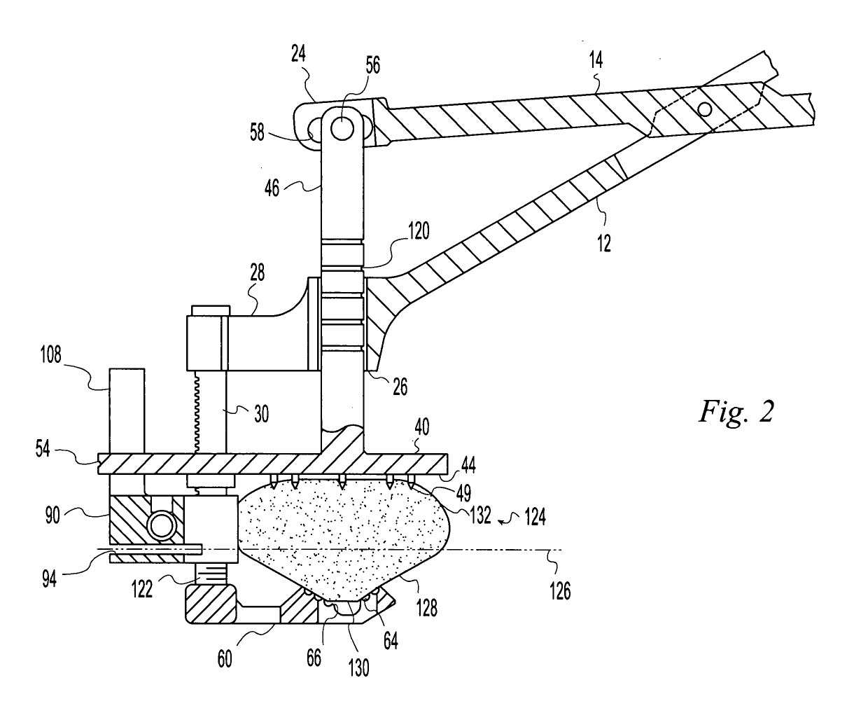

In use (FIG. 2), a patella 124 is clamped between the anterior and posterior clamp faces 44, 64. The total patellar thickness may be read from the scale 120 on the support rod 46. In some procedures, the user may only need to determine the total patellar thickness to determine the amount of bone that should be removed or the amount of bone that should be left for a particular patellar implant or procedure. The user can then use the total patellar thickness information as needed. For example, after measuring the patella 124 using the scale 120 on the support rod 46, the patella 124 may be cut using any available instruments and techniques. However, in most procedures, it is desirable to also use the cut guide 90 to cut the patella. The cut guide 90 is adjusted vertically to position the cut plane 126 to a position corresponding to the desired bone remaining as indicated on the scale 116 on the cut guide 90 and/or to a position corresponding to the desired bone removed as indicated on the scale 122 on the guide rods 30. Once the appropriate height is set, a cutter is guided by the cut guide 90 to resect the patella 124 along the cut plane 126.

The posterior clamp member 60 shown in FIGS. 1-2 orients the patella 124 between the clamp faces by referencing the posterior clamp face 64. The shape of the posterior clamp face 64 resembles the articular surface of the posterior side 128 the patella 124. The patella 124 is located medially-laterally such that the patellar ridge 130 is located at the bottom of the curved clamp surface. Elongated teeth 66 grip the posterior side 128 of the patella 124 and maintain the orientation of the patella relative to the posterior clamp face 64 when the anterior clamp face 44 is tightened against the anterior side 132 of the patella 124. The anterior projections 49 prevent the patella 124 from slipping relative to the anterior clamp face 44.

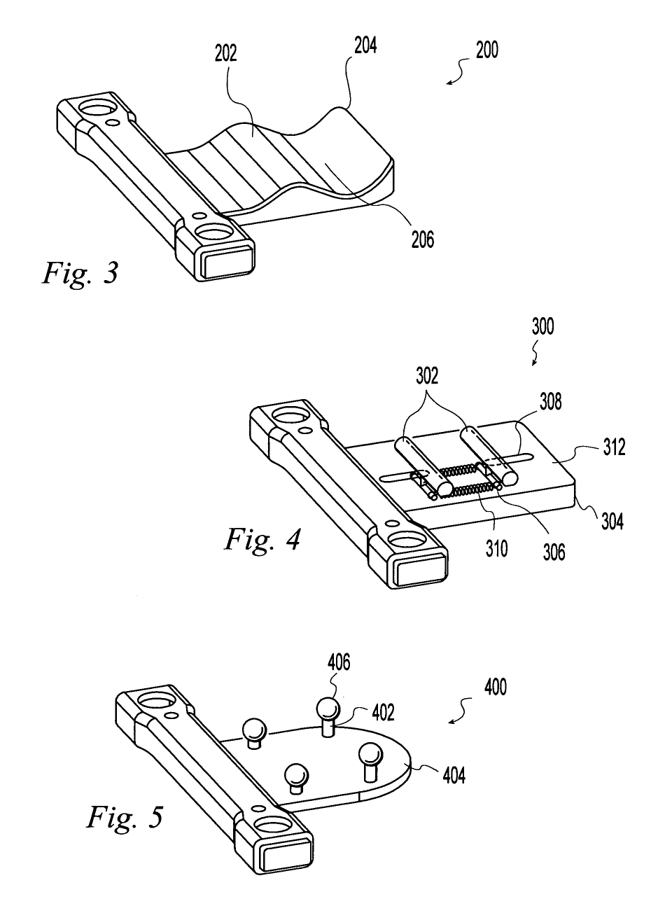

FIG. 3 illustrates an alternative posterior clamp member 200 that can be substituted for the posterior clamp member 60 of FIGS. 1 and 2. The alternative posterior clamp member 200 permits the anterior clamp face 44 to orient the patella 124. The alternative posterior clamp member 200 includes two humps 202, 204 defining a smooth trough 206 running superiorly-inferiorly between them. In use, the patella 124 is located medially-laterally such that the patellar ridge is seated at the bottom of the curved clamp surface in the trough 206. The humps 202, 204 contact the patellar facets and keep the patella located medially-laterally while the anterior clamp face 44 is pressed against the anterior side 132 of the patella 124. As the anterior clamp face 44 presses on the patella 124 the patella rotates on the smooth posterior clamp face until the anterior surface of the patella is generally parallel to the anterior clamp face 44 such that the patella is oriented relative to the anterior side 132 of the patella 124.

FIG. 4 illustrates an alternative posterior clamp member 300 that can be substituted for the posterior clamp member 60 of FIGS. 1 and 2. The alternative posterior clamp member 300 is similar to the alternative posterior clamp member of FIG. 3 inasmuch as it includes smooth humps that permit the patella to orient itself to the resection guide 10. However, the posterior clamp member 300 of FIG. 4 includes a pair of moveable humps able to translate toward and away from one another medially-laterally to adjust to the size of the patella. The humps are defined by cylindrical members 302 each mounted to the base 304 by way of a “T”-post 306 captured in a slot 308. The cylindrical members 302 are biased toward one another by a spring 310. In the illustrative embodiment, the cylindrical members 302 are also able to rotate relative to the base 304 and are connected to one another by a pair of springs that biases them toward one another and into parallel alignment. In use, as the patella is pressed into contact with the cylindrical members 302, they adjust their spacing and orientation to match the contours of the patella. The cylindrical members 302 may be mounted so that they can move apart enough for the patella to contact the surface 312 of the base 304 while the cylindrical members 302 help to support the patella. The surface 312 acts as a fixed reference from which accurate bone removal measurements may be gauged.

FIG. 5 illustrates an alternative posterior clamp member 400 that can be substituted for the posterior clamp member 60 of FIGS. 1 and 2. The alternative posterior clamp member 400 includes a plurality of posts 402 mounted to a base 404. The posts may optionally include spherical heads 406. The posts 402 contact the patellar facets to orient the patella in a desired orientation relative to the patella clamp 10. In one configuration the posts 402 are permanently set at predetermined heights. In another configuration the posts 402 are manually adjustable such as by screwing the posts 402 into or out of the base 404 to adjust the relative heights of the posts 402 and thus the patellar orientation. In another configuration, the posts 402 are vertically spring loaded so that they adjust automatically to the contours of the patella during clamping.

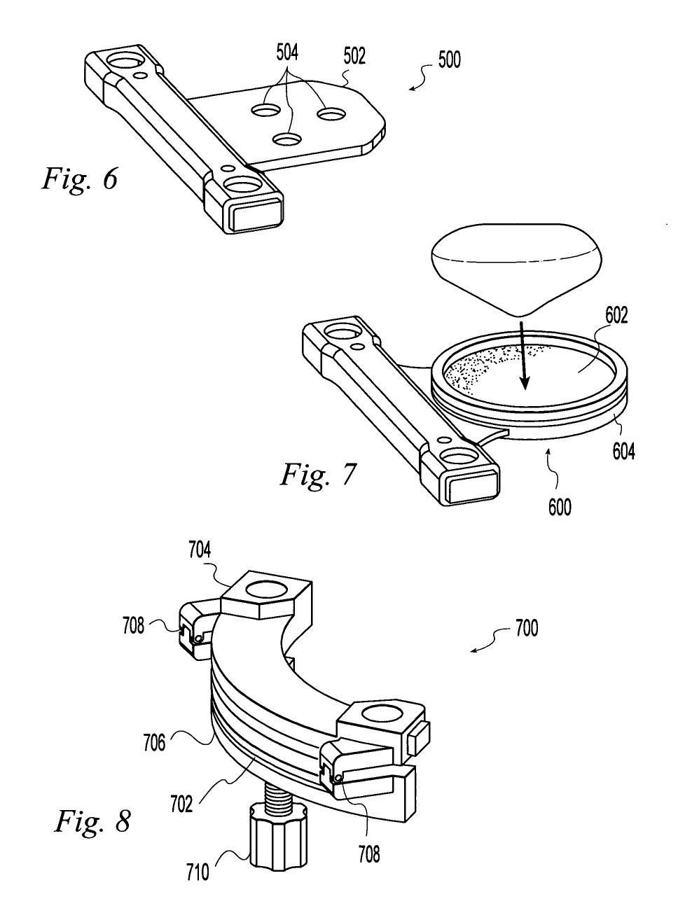

FIG. 6 illustrates an alternative posterior clamp member 500 that can be substituted for the posterior clamp member 60 of FIGS. 1 and 2. The alternative posterior clamp member 500 includes a relatively thin, flat, rigid base 502. The alternative posterior clamp member 500 can be used as a patellar protector to protect the resected posterior surface of the patella during subsequent steps of the surgical procedure. For example, this is useful in some minimally invasive surgical procedures in which the patella is cut first and then the remaining joint compartments are cut. In one exemplary surgical technique, after the patella has been resected, the cut guide 90 and posterior clamp member 60 may be removed and replaced with the alternative posterior clamp member 500. The patella clamp 10 may then be reapplied to the patella such that the base 502 covers the resected patellar surface. The patella clamp 10 may also be used to retract and otherwise manipulate the patella while subsequent surgical steps are carried out. The base 502 may optionally include guide openings in the form of holes, slots, notches, and/or other shapes to guide subsequent forming of the patella to receive an implant. For example, in the illustrative embodiment, drill guide holes 504 are formed through the base 502 to guide a drill bit to form peg receiving holes in the patella.

FIG. 7 illustrates an alternative posterior clamp member 600 that can be substituted for the posterior clamp member 60 of FIGS. 1 and 2. The alternative posterior clamp member 600 is useful to protect an unresected patella during subsequent surgical steps. Some surgeons prefer not to resurface the patella during a knee replacement operation. In such cases it is important to protect the natural articular surface of the patella. In order to accommodate minimally invasive procedures, the device is made very thin to minimize its intrusion into the surgical field. The exemplary device includes a thin membrane 602 of durable, flexible material that is placed over the posterior surface of the patella. In the exemplary embodiment, the membrane 602 is sandwiched between a pair of rings 604. Different ring diameters may be provided to accommodate differently sized patellae. The rings may be circular, ovular, irregular, or any other suitable shape. The membrane may be made of polymers, metals, and/or other suitable materials. Preferably, the membrane is made of an elastomer that can stretch around the patella. An example of a suitable material that has been used in surgical drapes used with powered instruments is Texin® 5286. In use, the alternative posterior clamp member 600 is attached to the patella clamp 10 and clamped over the patella such that the membrane is pulled up tightly over the posterior surface of the patella.

FIG. 8 illustrates an alternative cut guide 700 that can be substituted for the cut guide 90 of FIGS. 1-2. The alternative cut guide 700 is adjustable to change the orientation of the cut slot 702. The cut guide 700 includes a first member 704 engageable with the guide rods 30 in height adjustable relationship to allow the vertical position of the cut plane to be adjusted relative to the patella. The cut guide 700 includes a second member 706 defining the cut slot 702 and mounted to the first member in angular adjustable relationship. In the illustrative embodiment, the second member 706 is connected to the first member 704 by a pair of hinges 708. An adjustment screw 710 threadably engages the second member 706 and is captured by the first member 704 such that rotation of the adjustment screw 710 pivots the second member 706 about the hinges 708 relative to the first member and thereby changes the angular orientation of the cut slot 702. Preferably the angle of the cut slot is marked on the adjustment screw to permit adjusting the cut slot to predetermined angular values. The illustrative cut guide is adjustable to vary the medial-lateral angle of the cut plane. The cut guide may be adjustable to vary the medial-lateral, anterior-posterior, and/or any other angle. The illustrative hinges 708 are shown as a three-piece pinned assembly. Alternatively, the hinges 708 may be a single-piece such as a leaf spring or living hinge arrangement.

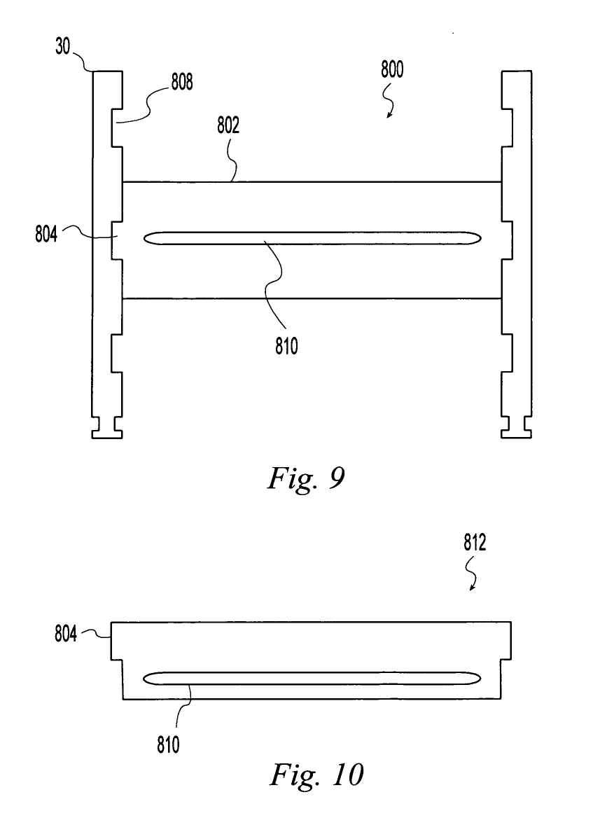

FIG. 9 illustrates an alternative cut guide 800 that can be substituted for the cut guide 90 of FIGS. 1-2. The alternative cut guide 800 includes a symmetric block body 802 having ears 804 extending from opposite sides of the body 802. The guide rods 30 of the patella clamp 10 include notches 808 to receive the ears 804. The cut guide 800 can be alternatively positioned in different notches 808 to adjust the cut plane position vertically. A cut guide slot 810 guides a cutter in the cut plane. FIG. 10 illustrates an offset cut block body 812 having a cut guide slot 810 offset from the ears 804 to provide cut plane adjustment increments between those provided by the notches 808 in the guide rods 30. By substituting the offset cut block body 812 for the symmetric cut block body, the cut slot 810 height is offset in one direction to an intermediate value between two notches 808. The offset cut block body 812 can be inverted to provide an offset in the opposite direction.

Although examples of a patella clamp 10 and its use have been described and illustrated in detail, it is to be understood that the same is intended by way of illustration and example only and is not to be taken by way of limitation. The invention has been illustrated in use to clamp the patella relative to a cut guide during a surgical procedure on the knee. However, the patella clamp 10 may be used with or without the cut guide as a patellar clamp, retractor, cut guide, protector, drill guide, and in other uses as are consistent with the physical characteristics of the device. Accordingly, variations in and modifications to the patella resection guide and its use will be apparent to those of ordinary skill in the art, and the following claims are intended to cover all such modifications and equivalents.

1. A patella clamp for clamping a patella having first and second surfaces, the patella clamp comprising:
a first clamp member engageable with the first patellar surface;
a second clamp member removably mountable in opposing relative moving relationship to the first clamp member, the second clamp member being engageable with the second patellar surface; and
a third clamp member alternatively mountable in place of the second clamp member to change the function of the patella clamp.

2. The patella clamp of claim 1 wherein the second clamp member includes means for gripping the second patellar surface to orient the patella in the patella clamp relative to the second patellar surface.

3. The patella clamp of claim 1 wherein the second clamp member includes means for slidably supporting the second patellar surface to orient the patella in the patella clamp relative to the first patellar surface.

4. The patella clamp of claim 1 wherein the second clamp member includes means for protecting the posterior patellar surface during subsequent surgical steps.

5-8. (canceled)

9. The patella clamp of claim 1 further comprising:
a cut guide mounted relative to the first and second clamp members, the cut guide including means for guiding a cutter in a cut plane to resect a portion of the patella.

10. The patella clamp of claim 9 wherein the cut guide is adjustable relative to the first and second clamp members to vary the cut plane location relative to the patella.

11-13. (canceled)

14. The patella clamp of claim 1 further comprising a scale that indicates the spacing of the first and second clamp members and thus the total patella thickness.

15. The patella clamp of claim 9 further comprising a first scale that indicates the spacing of the first clamp member from the cut plane and a second scale that indicates the spacing of the second clamp member from the cut plane such that the first and second scales indicate the patella thickness that will remain after cutting the patella and the amount of patellar bone that will be removed during cutting of the patella.

16. A patella clamp for clamping a patella, the patella having an anterior side and a posterior side, the posterior side having a central patellar ridge and medial and lateral facets, the patella clamp comprising:
a handpiece having first and second handles pivotably mounted together, the handles including a grip end and a working end;
an anterior clamp member including a base defining an anterior clamp face, the anterior clamp member being mounted to the working end of the first handle in translating relationship, the anterior clamp member being pivotably connected to the working end of the second handle;
a first posterior clamp member including a base defining a posterior clamp face, the first posterior clamp member being removably mounted to the working end of the first handle in opposing relationship to the anterior clamp member such that moving the grip ends of the first and second handles toward and away from one another causes the anterior clamp member to translate toward and away from the first posterior clamp member; and
a second posterior clamp member including a base defining a posterior clamp face, the second posterior clamp member being intraoperatively interchangeable with the first posterior clamp member to modify the function of the patella clamp.

17. The patella clamp of claim 16 further comprising:
a cut guide defining a cut plane and able to guide a cutter in the cut plane, the cut guide being mounted to the working end of the first handle for translation relative to the anterior and posterior clamp members, the cut guide including a surface defining a scale; and
a pointer mounted to the anterior clamp member adjacent to the cut guide scale, the pointer and scale indicating the distance from the anterior clamp face to the cut plane.

18. The patella clamp of claim 17 wherein the anterior clamp member includes a stop against which the cut guide abuts to prevent the cut guide from being adjusted closer than a predetermined minimum distance from the anterior clamp face and thus to establish a minimum remaining patella thickness.

19. (canceled)

20. The patella clamp of claim 17 wherein the anterior clamp member further comprises a scale readable relative to the working end of the first handle to indicate the distance between the anterior and posterior clamp faces.

21. The patella clamp of claim 17 wherein the working end of the first handle further comprises a scale readable relative to the cut guide to indicate the distance from the posterior clamp face to the cut plane.

22. The patella clamp of claim 16 wherein one of the first and second posterior clamp members defines a curved clamp face including teeth engageable with the posterior side of the patella in non-sliding engagement.

23. The patella clamp of claim 16 wherein one of the first and second posterior clamp members has a clamp face including a pair of smooth humps defining a smooth trough between them, the humps being engageable with the medial and lateral facets of the posterior side of the patella while the trough receives the posterior patellar ridge, the humps being able to position the patella medially-laterally while allowing the patella to rotate such that the anterior surface of the patella is generally parallel to the anterior clamp face.

24. The patella clamp of claim 23 wherein the humps are mounted relative to one another for translation toward and away from one another medially-laterally to adjust to the size of the patella.

25. (canceled)

26. The patella clamp of claim 16 wherein one of the first and second posterior clamp members comprises a plurality of posts mounted to a base, the posts being engageable with the facets to orient the patella in a desired orientation relative to the patella clamp.

27. The patella clamp of claim 26 wherein the posts are adjustable relative to the base to vary the patellar orientation.

28. The patella clamp of claim 27 wherein the posts are vertically spring loaded such that they adjust automatically to the contours of the patella during clamping.

29. The patella clamp of claim 16 wherein one of the first and second posterior clamp members comprises a relatively thin, flat base defining a protective cover for the patella.

30. (canceled)

31. The patella clamp of claim 16 wherein one of the first and second posterior clamp members comprises a thin membrane conformable to the posterior patella during clamping.

32. (canceled)

33. The patella clamp of claim 17 wherein the cut guide includes a first member engageable with the working end of the first handle in height adjustable relationship to allow the vertical position of the cut plane to be adjusted relative to the patella, the cut guide further including a second member defining the cut plane and being mounted to the first member in angular adjustable relationship.

34. (canceled)

35. The patella clamp of claim 17 wherein the cut guide comprises a first body having ears extending from opposite sides of the body and the working end of the first handle defines a plurality of notches to receive the ears, the cut guide being alternatively positionable in the plurality of notches to adjust the cut plane position vertically.

36. The patella clamp of claim 35 further comprising a second, alternative cut guide body defining an offset cut plane, the offset cut guide being alternatively engageable with the plurality of notches to provide cut plane adjustment increments between those provided by the first body.

37. A patella clamp for clamping a patella, the patella having an anterior side and a posterior side, the posterior side having a central patellar ridge and medial and lateral facets, the patella clamp comprising:
a handpiece having first and second handles pivotably mounted together, the handles including a grip end and a working end;
an anterior clamp member including a base defusing an anterior clamp face, the anterior clamp member being mounted to the working end of the first handle in translating relationship, the anterior clamp member being pivotably connected to the working end of the second handle;
a posterior clamp member including a base defining a posterior clamp face, the posterior clamp member being mounted to the working end of the first handle in opposing relationship to the anterior clamp member such that moving the grip ends of the first and second handles toward and away from one another causes the anterior clamp member to translate toward and away from the posterior clamp member; and
cut guide mounted to the patella clamp and defining a transverse cut plane, the cut guide being able to guide a cutter transversely between the anterior and posterior clamp members to cut the patella in the cut plane, the cut guide and at least one of the anterior and posterior clamp members defining a scale between them indicating the position of the cut plane relative to at least one of the anterior and posterior clamp faces.
